(19) United States
(12) Patent Application Publication
(10) Pub. No.: US 2012/0222677 A1
(43) Pub. Date: Sep. 6, 2012

(54) NOVEL SELF-INFLATING BAG WITH SPECIFIC COMPRESSION MARKS FOR PEDIATRIC AND ADOLESCENT RESUSCITATION

(75) Inventors: Kyeong Ryong Lee, Seoul (KR); Sang O. Park, Gyeonggi-do (KR); Kwang Je Baek, Seoul (KR); Dae Young Hong, Seoul (KR)

(73) Assignee: Konkuk University Industrial Cooperation Corporation, Seoul (KR)

(21) Appl. No.: 13/224,508

(22) Filed: Sep. 2, 2011

(30) Foreign Application Priority Data
Mar. 3, 2011 (KR) 10-2011-0018981

(51) Int. Cl. A61M 16/08

(52) U.S. Cl. 128/205.14

(57) ABSTRACT

There is provided a novel self-inflating bag with specific compression marks for pediatric and adolescent resuscitation. The self-inflating bag provides various different range of accurate tidal volume depending on different size or weight of patient by marking compression points on the self-inflating bag.

### CROSS-REFERENCE TO RELATED APPLICATION

This application claims under 35 U.S.C. §119(a) the benefit of Korean Application No. 10-2011-0018981 filed Mar. 3, 2011, the entire contents of which are incorporated herein by reference.

### TECHNICAL FIELD

The present invention relates to a novel self-inflating bag with specific compression marks for pediatric and adolescent resuscitation.

### BACKGROUND ART

The self-inflating bag is regarded as the standard method for temporary respiratory support, including during sudden cardiopulmonary arrest. However, manual use of bag ventilation leads to delivery of a very wide range of tidal volume (hereinafter referred to as ‘TV’), due to variable compressions of the bag by rescuers who have varying squeeze methods and who differ in individual physical characteristics such as hand size, volume and grip power. (Lee H M, Cho K H, Choi Y H, Yoon S Y, Choi Y H. Emerg Med J 2008; 25:632-4; Elling R, Politis J. Ann Emerg Med 1983; 12:765-8; Carden E, Hughes T. Anesth Analg 1975; 54:133-8; Augustine J A, Seidel D R, McCabe J B. Am J Emerg Med 1987; 5:267-70; McCabe S M, Smeltzer S C. Am J Crit Care 1993; 2:467-73; Koga T, Kawamoto M. J Clin Anesth 2009; 21:178-82)

Therefore, manual use of this bag may have limitations in delivering accurate TV. Respiratory arrest accounts for a large proportion of pediatric arrests. Early recognition and prompt treatment of respiratory compromise is critical for improving survival and preventing progression to cardiac arrest. Delivery of adequate oxygen and removal of carbon dioxide is an essential requirement in most pediatric victims. (Young K D, Gausche-Hill M, McClung C D, Lewis R J. Pediatrics 2004; 114:157-64; Schindler M B, Bohn D, Cox P N, et al. N Engl J Med 1996; 335:1473-9).

However, because children have variable body size, weight and lung capacity, variable ranges of accurate TV delivery might be required. It is difficult to deliver an adequate TV to children of varying size, requiring various sizes of bag for accurate delivery. In the 2005 pediatric basic life support guidelines, only 450-500 ml self-inflating bags were recommended, and the method of delivering accurate ventilations to large children and adolescents using a bag was not well described. (International Liaison Committee on Resuscitation. 2005 International consensus on cardiopulmonary resuscitation and emergency cardiovascular care science with treatment recommendations. Part 6. Pediatric basic and advanced life support. Resuscitation 2005; 67:271-91)

Rescuers might not deliver precise TV to children with different size and lung capacity, using either a pediatric bag or an adult bag, and inspection of visible chest rise by rescuers was the only guidance to support adequate TV delivery. (International Liaison Committee on Resuscitation. 2005 International consensus on cardiopulmonary resuscitation and emergency cardiovascular care science with treatment recommendations. Part 6. Pediatric basic and advanced life support. Resuscitation 2005; 67:271-91)

In situations where victims had an advanced airway, chest rise could not be inspected by the bag compressor when ventilation was delivered, because another rescuer must perform closed chest compression without interruptions. Approximately 6-7 ml/kg is stated to be the volume of visible chest rise. (International Liaison Committee on Resuscitation. 2005 International consensus on cardiopulmonary resuscitation and emergency cardiovascular care science with treatment recommendations. Part 2. Adult basic life support. Resuscitation 2005; 67:187-201.) However, accurate TV delivery using this estimate may be impossible in real arrest situation with manual use of the bag. Inaccurate ventilation is closely related to poor patient outcomes in pediatric arrest and critical emergency situations. Too low volume will worsen hypoxia and acidosis and too large volume might compromise blood flow, interfere with circulation, and cause air trapping and barotrauma. (Guidelines 2000 for cardiopulmonary resuscitation and emergency cardiovascular care. Part 9. Pediatric basic life support. The American heart association in collaboration with the International Liaison Committee on resuscitation. Circulation 2000; 102:1253-90; Aufderheide T P, Sigurdsson G, Pirrallo R G, et al. Hyperventilation induced hypotension during cardiopulmonary resuscitation. Circulation 2004; 109:1960-5) Pediatric arrest might occasionally occur in a place in which pediatric resuscitation is a rare event, and it is unlikely that pediatric resuscitation equipment will be available, which necessitates that an adult bag be used for respiratory support.

### SUMMARY OF THE DISCLOSURE

The present invention is designed to solve the problems of the prior art, and therefore it is an object of the present invention to provide a novel self-inflating bag for pediatric and adolescent resuscitation.

According to an aspect of the present invention, there is a self-inflating bag for pediatric and adolescent resuscitation providing various different range of accurate tidal volume depending on different size or weight of patient by marking compression points on the self-inflating bag.

### 1) Novel Adult Self Inflating Bag (TVMB)

The self-inflating bag according to one exemplary embodiment of the present invention wherein in the self-inflating adult bag, equally dividing the bag into five parts from the start point to mid-point of the bag and setting point 1, point 2, point 3 and point 4 in order except mid-point on the surface of the bag from a respiratory valve of the bag, thereby providing 100-200 ml tidal volume at point 1 near the valve, 200-300 ml tidal volume at point 2, 300-400 ml tidal volume at point 3, and 400-500 ml tidal volume at point 4 The present invention encompasses a method providing accurate tidal volume depending on different size or weight of pediatric and adolescent patients comprising: preparing a self-inflating adult bag with specific compression marks on the surface of the bag and providing 100-200 ml tidal volume at point 1 near the valve, 200-300 ml tidal volume at point 2, 300-400 ml tidal volume at point 3, and 400-500 ml tidal volume at point 4, by equally dividing the bag into five parts from the start point to mid-point of the bag and setting point 1, point 2, point 3 and point 4 in order except mid-point on the surface of the bag from a respiratory valve of the bag in the self-inflating adult bag.

### 2) Novel Pediatric Self Inflating Bag

The self-inflating bag according to another exemplary embodiment of the present invention wherein in the self-inflating pediatric bag, equally dividing the bag into four parts from the start -point to mid-point of the bag and setting point 1, point 2, point 3 and point 4 in order on the surface of the bag from a respiratory valve of the bag, thereby providing 36-70 ml tidal volume at point 1 near the valve, 60-105 ml tidal volume at point 2, 90-168 ml tidal volume at point 3, and 144-210 ml tidal volume at point 4.

The method according to one exemplary embodiment of the present invention wherein in the self-inflating pediatric bag, providing 36-70 ml tidal volume at point 1 near the valve, 60-105 ml tidal volume at point 2, 90-168 ml tidal volume at point 3, and 144-210 ml tidal volume at point 4 by equally dividing the bag into four parts from the start point to mid-point of the bag and setting point 1, point 2, point 3 and point 4 in order on the surface of the bag from a respiratory valve of the bag.

3) Invention and Method of using Novel Self-Inflating Bag

The surface of the bag has compression points which are engraved in the surface of the bag or are embossed on the surface of the bag. In a specific embodiment of the present invention, the compression points are attached to the bag in the type of embossing with nonslip material.

The method according to another exemplary embodiment of the present invention wherein the method further comprising: placing thumb at the upper points of the bag and index or middle finger at the lower points of the bag; and compressing the bag until the upper and lower points meet one another over 1 second, to deliver preset target volumes.

### 4) Testing of Novel Adult Self Inflating Bag (TVMB)

Fifty-three subjects (28 doctors, 17 nurses, 8 paramedics) participated in this simulation trial. TVMB, pediatric bag and adult bag were connected to a gas flow analyzer for measuring TV and peak inspiratory pressure (PIP). In a random cross-over setting, participants delivered 10 ventilations using the adult bag, pediatric bag or TVMB in each of four target volume ranges (100-200 ml, 200-300 ml, 300-400 ml, 400-500 ml). We compared TV and PIP for the adult bag, pediatric bag and TVMB in each subject. Compared with the pediatric bag, TVMB showed higher rates of accurate TV delivery in the 200-300 ml target volume range (87-90% versus 32-35%; p<0.05). Compared with the adult bag, TVMB showed higher rates of accurate TV delivery in all target volume ranges (75-90% versus 45-50%; p<0.05).

The frequency of too high or low TV delivery was higher with the adult bag than TVMB (20-30% versus 0-5%; p<0.05). There was no significant difference in PIP between the pediatric bag and TVMB (within 5 cm H2O; p<0.05).

Therefore, TVMB could deliver accurate TV in various target volume ranges for pediatric and adolescent resuscitation.

### 5) Testing of Novel Pediatric Self Inflating Bag

In addition, to compare the accuracy of manually delivered target tidal volumes (TV) with the conventional pediatric self-inflating bags (CPB) versus the novel pediatric self-inflating bags (NPB) in children of varying weights.

Before the trial begun, four target TV ranges were established using the Broselow™ Tape as a reference: 36-70 ml for 6-10 kg, 60-105 ml for 10-15 kg, 90-168 ml for 15-24 kg, and 144-210 ml for 24-30 kg. A NPB with four surface marks matching the target TV ranges was prepared. Senior medical students (n=73) were enrolled. After one week of training in TV delivery with both CPB and NPB, subjects participated in a test simulation. Using the CPB and NPB in a random cross-over design, participants delivered 10 ventilations to test lungs connected to gas flow analyzers for the randomly assigned target TV ranges.

Each of the 730 values for TV and peak inspiratory pressures (PIP) delivered by CPB and NPB were analyzed. The proportion of accurate TV delivery was higher with NPB than with CPB: 84.2% versus 45.9% for 36-70 ml, 93.2% versus 42.7% for 60-105 ml, 96.0% versus 70.3% for 90-168 ml, and 91.2% versus 62.6% for 144-210 ml respectively (all p<0.001).

Compared to NPB, CPB delivery was more varied and was more frequently out of range. There were no significant differences in PIP between the CPB and NPB.

Therefore, NPB is useful as a ventilation device for the accurate delivery of TV to small children of varying weights.

### BRIEF DESCRIPTION OF DRAWINGS

FIG. 1 shows (A) newly designed target volume marked adult bag (TVMB) and (B) compression of Point 2 to deliver 200-300 ml target volume using the TVMB.

FIG. 2 shows schematic representation of the newly designed target volume marked adult bag (TVMB). Four points (Points 1-4) were marked on the surface of the adult bag, and imaginary bags for each point were simulated.

FIG. 3 shows box plots of delivered tidal volumes for target volumes. Comparisons are shown between the pediatric bag (PB) and the target volume marked bag (TVMB), and between the adult bag (AB) and the TVMB. Box plots represent the median values with upper and lower inter-quartile ranges. Square line boxes represent target volumes.

FIG. 4 shows box plots of delivered peak inspiratory pressures for each target volume. Comparisons shown are between the pediatric bag (PB) and the target volume marked bag (TVMB), and between the adult bag (AB) and the TVMB. Box plots represent the median value with upper and lower inter-quartile ranges.

FIG. 5 is schematic representation of the design process for the novel pediatric self-inflating bag. To determine the correct compression surface points, all nine available points on the surface of the pediatric bag (A) were prepared. After the testing procedure, of the nine compression points tested, four points that matched the four target volume ranges were determined (B).

FIG. 6 shows top view (A) and side view (B) of the novel pediatric self-inflating bag (NPB). Broselow tape (C) for the clinical application of the NPB.

FIG. 7 is dot plots of the delivered tidal volumes (TVs) for each target volume for the conventional pediatric self-inflating bag (□) and the novel pediatric self-inflating bag (o) by weight: 36-70 ml for 6-10 kg (A), 60-105 ml for 10-15 kg (B), 90-168 ml for 15-24 kg (C), and 144-210 ml for 24-30 kg (D). In each graph, the upper and lower horizontal lines (-----) indicate the upper and lower limits for the value of the target TV range, and the middle horizontal line indicates the median value of the target TV ranges.

FIG. 8 is mountain plots for comparing the distribution patterns of percentiles (Y-axis) for the difference in the median target volume and delivered volume (X-axis) between the conventional pediatric self-inflating bag (Gray) and the novel pediatric self-inflating bag(black).

FIGS. 9a and 9b show top view (a) and side view (b) of respective marking point on the adult self-inflating bag.

FIGS. 10a and 10b show top view (a) and side view (b) of respective marking point on the pediatric self-inflating bag.

### DETAILED DESCRIPTION OF THE DISCLOSURE

Hereinafter, non-limiting embodiments of the present invention will be described in more detail with reference to the accompanying drawings.

### 1) A Study of Novel Adult Self-Inflating Bag (TVMB)

### (1) Method

This was a simulation trial with a randomized controlled crossover design. Our study protocol was reviewed and approved by the Institutional Review Board for Human Research at Konkuk University Hospital.

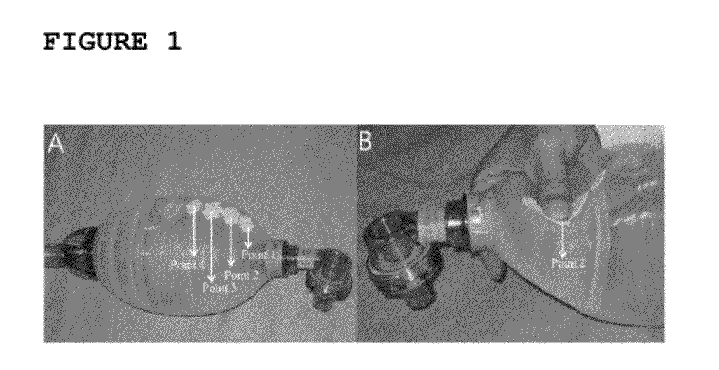

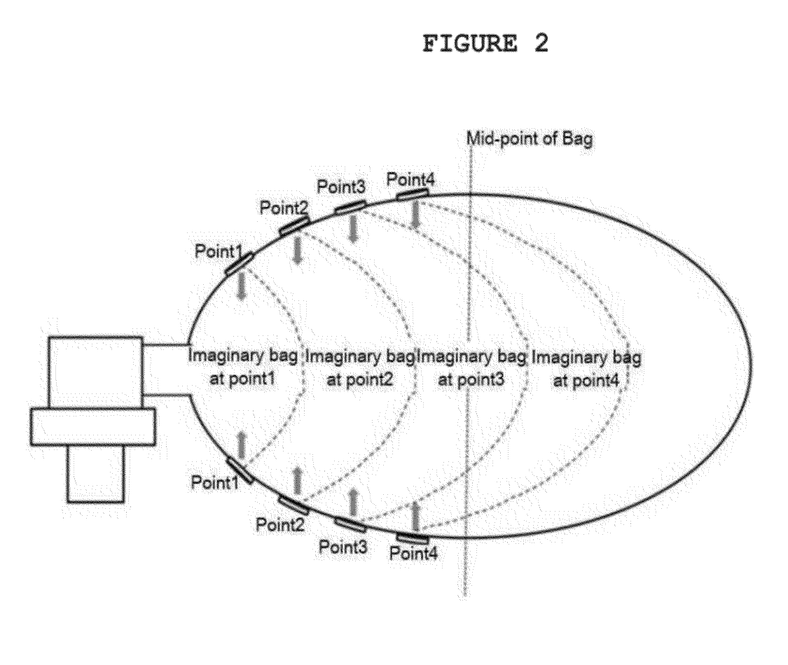

We designed an adult self-inflating 1600 ml bag with four compression points marked on the resuscitator bag surface (FIG. 1A). The TV delivered by the bag was related to the bag's maximum volume. We determined the maximum volumes for each mark using an imaginary bag. For each of the four points, four imaginary bags were simulated (FIG. 2). The maximum volume for the four imaginary bags was measured using the following procedure.

Water was poured into the bag to the line of each point and this water was transferred into a water meter bottle. Doubling of the volume was accepted as the maximum volume for the four imaginary bags (160 ml, 350 ml, 750 ml and 880 ml, respectively).

Generally, one third to two thirds of the bag's maximum volume was known to be delivered as the TV. Thus, we roughly estimated the volume ranges delivered by squeezing the bag at each point, accordingly (Point 1: 100-200 ml, Point 2: 200-300 ml, Point 3: 300-400 ml and Point 4: 400-500 ml). Subjects compressing the bags were instructed to place their fingers at the upper and lower points and compress the bag until the upper and lower points met one another, to deliver preset target volumes (FIG. 1B).

The TVMB, pediatric 500 ml bag and adult 1600 ml bag were connected to a gas flow analyzer (VT PLUS HFTM, Fluke Biomedical, Everett, Wash., USA) for measurement and display of the TV and peak inspiratory pressure (PIP). This device was connected to a test lung for physiological simulation of ventilation and all data were recorded using a Vent tester program (Version 2.01, Fluke Biomedical, Everett, Wash., USA) with a laptop computer connected to the flow analyzer.

Doctors, nurses and paramedics from the emergency department, pediatric intensive care unit and dental clinics were recruited for this study. All participants were certified in basic life support. After obtaining informed consent, brief instructions were given for using the pediatric bag, adult bag and the TVMB. Participants were randomly allocated to either ‘first conventional bag group’ or ‘first modified bag group’. During the study, participants and testers could not see the results of the TV and PIP. Participants in the ‘first conventional bag group’ were instructed to squeeze the pediatric bag or adult bag for over 1 s. Participants each made 10 ventilations using the one-handed bag squeezing technique with 6-8 s intervals, and 10 using the two-handed bag squeezing technique with 6-8 s intervals, in four different situations where target volumes were tested in a random order: 100-200 ml and 200-300 ml using both the pediatric bag and adult bag, 300-400 ml and 400-500 ml using only the adult bag.

Participants in the ‘first modified bag group’ were instructed to squeeze the TVMB using the same protocol as the ‘first conventional bag group’: 100-200 ml target, 200-300 ml target, 300-400 ml target and 400-500 ml target (using only the TVMB). After completing each process in two groups, all participants took a break for 1 h before performing the cross-over component of the study. Candidates who first participated in the ‘first conventional bag group’ performed the protocol using the TVMB, and candidates who first participated in the ‘first modified bag group’ performed the protocol using the conventional pediatric and adult bags. All TV and PIP data were recorded on a laptop computer and transferred to a spreadsheet (Microsoft Excel 2007, Microsoft Corporation, Seoul, Korea). To assess the usefulness of each bag device, the rate of accurate volume delivery and ‘too high or low’ volume delivery were assessed. ‘Too high or low volume’ was defined as 50 ml less than the normal lower limit, or 50 ml more than the normal upper limit.

We analyzed the data using statistical software (SPSS, version 17.0, SPSS Inc., Seoul, Korea). A paired t-test and chi-square test were used to test for differences between the two groups. Two-sided p values<0.05 were considered statistically significant.

Fifty-three volunteers participated in this simulation study: 28 doctors, 17 nurses and 8 paramedics. Participants were between 23 and 42 years old (mean age was 30.7±4.5) and 26 were male (49.1%).

### (2) Result 1; Conventional Pediatric Bag Versus the TVMB

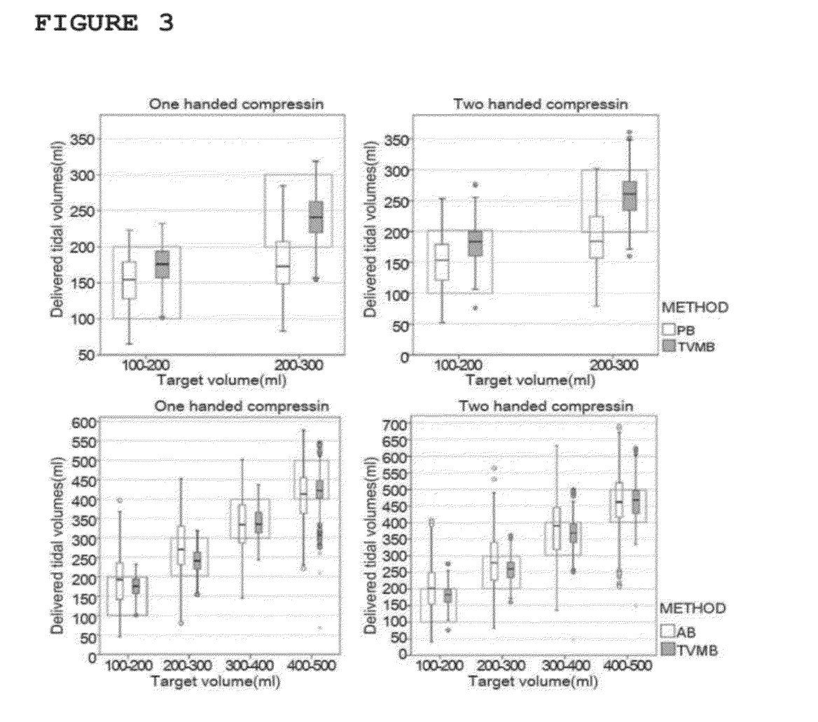

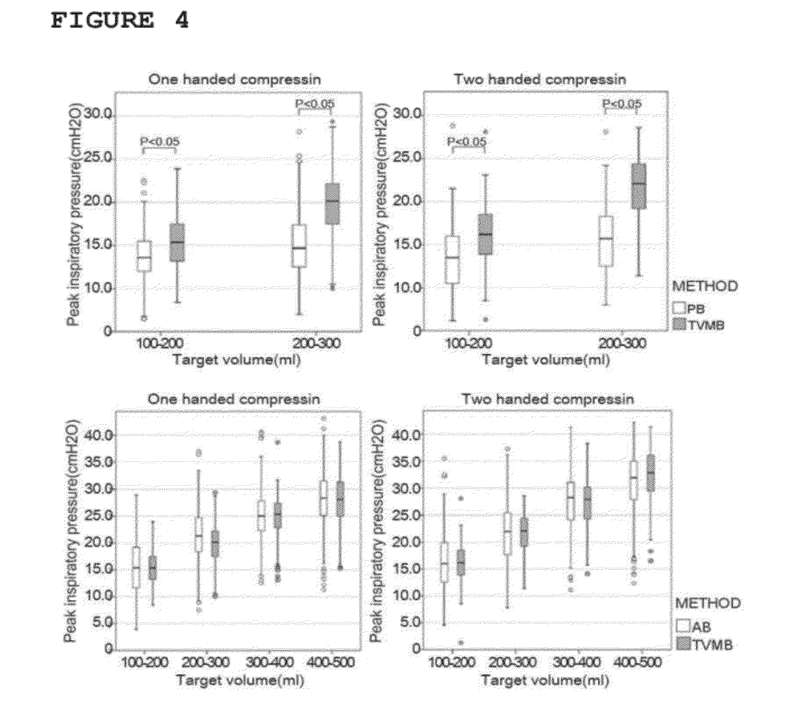

All median values with upper and lower quartile ranges of TV delivery using the TVMB were included in each target volume range, but those using the pediatric bag were outside the 200-300 ml target range (FIG. 3). TV delivery by the pediatric bag showed a lower rate of accurate TV (32.5% using the one-handed technique, 39.8% using the two-handed technique) and a higher rate of too high or low TV in pediatric bag use (25.5%, one-handed; 21.5%, two handed) (Table 1). Median values with upper and lower quartile ranges of PIP using the TVMB were higher than those using the pediatric bag. These differences were within 5 cm H2O (FIG. 4).

TABLE 1
The rate of accurate and too high or low tidal volume delivery between the paediatric bag and the target volume marked bag.

| | No of accurate TV delivery (%) | | | No of too high or low TV delivery (%) | | |
|---|---|---|---|---|---|---|
| Target volume (ml) | Paediatric bag | TVMB | p-Value | Paediatric bag | TVMB | p-Value |
| One hand compression | | | | | | |
| 100-200 | 448/530 (84.5) | 466/530 (87.9) | 0.130 | 0/530 (0) | 0/530 (0) | — |
| 200-300 | 172/530 (32.5) | 480/530 (90.6) | <0.001 | 135/530 (25.5) | 0/530 (0) | <0.001 |
| Two hand compression | | | | | | |
| 100-200 | 434/530 (81.9) | 422/530 (79.6) | 0.391 | 1/530 (0.2) | 4/530 (0.8) | 0.374 |
| 200-300 | 211/530 (39.8) | 465/530 (87.7) | <0.001 | 114/530 (21.5) | 3/530 (0.6) | <0.001 |

TV: tidal volume,
TVMB: target volume marked bag.

### (3) Result 2; Adult Bag Versus the TVMB

For the TVMB, all median values with upper and lower quartile ranges of delivered TV were included in the target volume ranges (FIG. 3). However, median values with upper and lower quartile ranges of delivered TV were outside target volumes in all target volume ranges for the adult bag, and showed a wider range of volume distribution than when using the TVMB (FIG. 3).

The rates of accurate TV delivery were higher with the TVMB (75-90%) than with the adult bag (35-50%), and the rate of too high or low TV delivery was higher with the adult bag (20-30%) than with the TVMB (0-5%) (Table 2). There were no differences in PIP between the adult bag and the TVMB (FIG. 4).

### (4) The Study Conclusion

From this simulation study, we found that our newly designed TVMB was able to deliver more accurate TV in various target volume ranges than the conventional pediatric bag or adult bag. The TVMB may be a promising device to deliver accurate TV during child and adolescent resuscitation.

TABLE 2
The rate of accurate and too high or low TV delivery between the Adult bag and the TVMB.

| | No of accurate TV delivery (%) | | | No of too high or low TV delivery (%) | | |
|---|---|---|---|---|---|---|
| Target volume (ml) | Adult bag | TVMB | p-Value | Adult bag | TVMB | p-Value |
| One handed compression | | | | | | |
| 100-200 | 253/530 (47.7) | 466/530 (87.9) | <0.001 | 93/530 (17.5) | 0/530 (0) | <0.001 |
| 200-300 | 257/530 (48.5) | 480/530 (90.6) | <0.001 | 125/530 (23.6) | 0/530 (0) | <0.001 |
| 300-400 | 270/530 (50.9) | 449/530 (84.7) | <0.001 | 84/530 (15.8) | 5/530 (0.9) | <0.001 |
| 400-500 | 275/530 (51.9) | 400/530 (75.5) | <0.001 | 107/530 (20.2) | 33/530 (6.2) | <0.001 |
| Two handed compression | | | | | | |
| 100-200 | 242/530 (45.7) | 422/530 (79.6) | <0.001 | 127/530 (24.0) | 4/530 (0.8) | <0.001 |
| 200-300 | 244/530 (46.0) | 465/530 (87.7) | <0.001 | 120/530 (22.6) | 3/530 (0.6) | <0.001 |
| 300-400 | 195/530 (36.8) | 427/530 (80.6) | <0.001 | 161/530 (30.4) | 13/530 (16.4) | <0.001 |
| 400-500 | 256/530 (48.3) | 393/530 (74.2) | <0.001 | 118/530 (22.3) | 23/530 (4.3) | <0.001 |

TV: tidal volume.
TVMB: career volume marked bag.

### 2) A Study of Novel Pediatric Self-Inflating Bag (NPB)

### (1) Designing and Manufacturing of the NPB

Determination of the Target TV Ranges in Small Children with Varying Weights

In the current guidelines for pediatric resuscitation, there is no reference table accurate tidal volumes based on body weight or age, and the visible chest rise paradigm is used only for guidance, rather than as an accurate and specific volume (Biarent D, Bingham R, Eich C, et al. European resuscitation council guidelines for resuscitation 2010 section 6. pediatric life support. Resuscitation. 2010 October; 81(10):1364-88; International Liaison Committee on Resuscitation. 2005 international consensus on cardiopulmonary resuscitation and emergency cardiovascular care science with treatment recommendations. part 6: Pediatric basic and advanced life support. Resuscitation. 2005; Guidelines 2000 for cardiopulmonary resuscitation and emergency cardiovascular care. part 9: Pediatric basic life support. the american heart association in collaboration with the international liaison committee on resuscitation. Circulation. 2000 Aug. 22; 102(8Suppl):1253-90. November-December; 67(2-3):271-91).

Generally, a TV of 6Generally, a TV of v-December; 67 (2-3):271-911-91 (2-3):271-91 (2-3) :271-91 August-2 (International Liaison Committee on Resuscitation. 2005 international consensus on cardiopulmonary resuscitation and emergency cardiovascular care science with treatment recommendations. part 2: Adult basic life support. Resuscitation. 2005 November-December; 67 (2-3):187-201; Baskett P, Nolan J, Parr M. Tidal volumes which are perceived to be adequate for resuscitation. Resuscitation. 1996 June; 31 (3):231-4.). Thus, we used the Broselow™ Pediatric Emergency Tape (Broselow tape) to determine the following four target TV ranges based on weight: 36uring fourhe following fouro be adequate for7 (2-3 colour in the Broselow Tape), 60ape), 60ape), 60e), 60: 36uring fourhe following four color in the Broselow Tape), 90ape), 90ape), 60e), 60: 36uring fourhe following fouro b in the Broselow Tape), and 144 and 144and 14460: 36uring fourhe following fouro be ade Broselow tape).

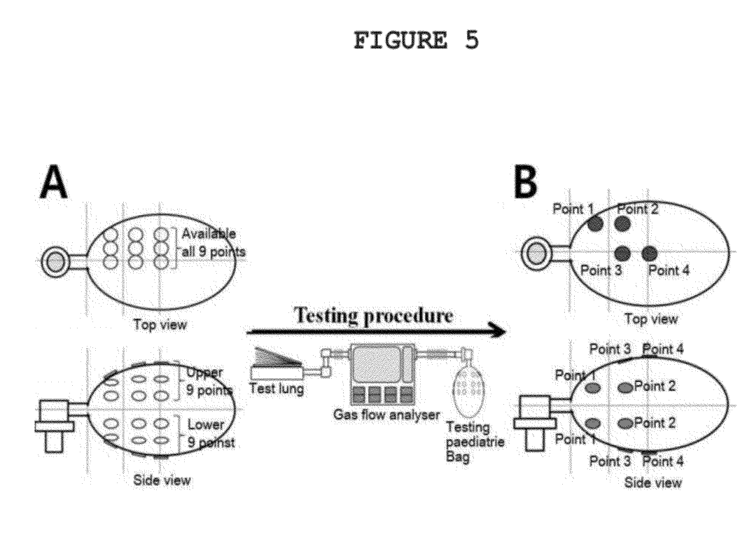

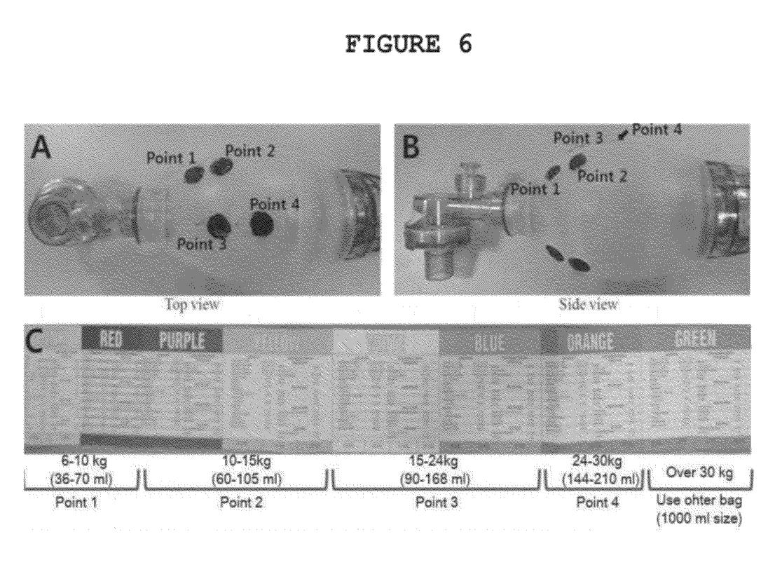

We assumed that compression at the same point on the surface of bag would deliver more steady TVs and that different points placed on the oval-shaped bag surface would allow for the delivery of different TVs. First, we designed nine available points that could be located by fingers on the surface of the pediatric self-inflating bag (FIG. 5A). The test pediatric self-inflating bag with nine compression points was connected to a gas flow analyzer (VT PLUS HFTM, Fluke Biomedical, Everett, Wash., USA), which was connected to a test lung on the other side. A total of 13 expert emergency and pediatric physicians participated in this testing process. Brief instructions for using the test pediatric self inflating bag were given to the 13 expert testers. Participants were instructed to place their fingers at each upper and lower point, and to compress the bag until the upper and lower points met one another over one second. In the testing process, each participant made 10 ventilations using the one-handed bag squeezing technique, with 6-8 sec intervals for each of the nine points. The total testing processes for the nine points were established, and 130 TV values (10 TV results for the 13 testers) for each of the nine points were collected and analyzed. Of the nine compression points, four compression points were determined to match well with each of the four target TV ranges. They are as follows: point 1 matched with the 36-70 ml range, point 2 matched with the 60-105 ml range, point 3 matched with the 90-168 ml range, and point 4 matched with the 144-210 ml range (FIG. 5B, FIG. 6).

### (2) Method: CPB Versus NPB; Randomised Cross-Over Simulation Study

### Study Design

This simulation trial used a prospective, randomized, and cross-over design. This simulation study was conducted at the simulation centre of basic life support education in our institution. Our study protocol was approved by the Institutional Review Board for Human Research at Konkuk University Hospital.

### Subjects

Between February 2011 and May 2011, a total of 11 education and simulation trials were established, and 6-7 healthy senior medical students participated in each course. A total of 73 senior medical students from our institution were enrolled in this study, of which 39(53%) were male. The subjectswith the 144-210 ml range (Fig. s) between the conve±2.1 years).

### Education and Testing

After written informed consent was obtained from each student, participants were educated about the use of both the CPB and the NPB. The education course consisted of 10 min of verbal instruction concerning the use of both pediatric self-inflating bags and a 40 min self-practice course. Students were then instructed to deliver TV using the one-handed bag squeezing technique over one second.

When participants performed the bagging themselves, they could see their real-time TV data on the screen of the gas flow analyzer, which was connected to the bags. Real-time self-feedback was given to all of the participants. They then practiced delivering all four target TVs (36was given to all of the participants. They then practiced the CPB and NPB until they were comfortable with their use. When subjects compressed the NPB, they were instructed to place their fingers on the upper and lower compression points and to then squeeze the bag until the upper and lower points met one another for over one second.

One week after the end of the education course, the participants revisited our simulation centre for the simulation testing. They were randomly allocated to group A (using the CPB; N=3 or 4) or group B (using the NPB; N=3 or 4). Prior to the testing procedures.

We prepared a gas flow analyzer, which was connected to a test lung, and each pediatric self inflating bag (the CPB for group A and the NPB for group B) was connected to the analyzer device. Additionally, a laptop computer was connected to this gas flow analyzer for data storage. To avoid biased performances in bagging, four different scenarios (target volumes ranges were 36oid biased performances in bagging, four different scenarios (target volume a random order. Based on the randomly requested target TV ranges, participants squeezed the assigned pediatric self-inflating bag (the CPB for group A and the NPB for group B). The subjects compressed the assigned pediatric self-inflating bag for over one sec and supplied 10 ventilations at 6-se intervals. During this testing procedure, participants and test observers could not see the screen that showed the TV and PIP data. After all subjects in both groups completed each testing process, participants switched groups (i.e., participants in Group A switched to B and those in Group B switched to A). They were given a 1-h break and were then asked to repeat the same testing protocol with the group-appropriate self-inflating bag.

### Data Collection and Analysis

The primary outcome measures for our study were TV and PIP. After all testing procedures were completed, all TV and PIP data were collected in a spread sheet program (Microsoft Excel 2007, Microsoft Corporation, Seoul, Korea) and stored in a Vent tester program (Version 2.01, Fluke Biomedical). Data were analyzed using statistical software (SPSS version 17.0, SPSS, Inc., Seoul, Korea). Mountain plots were used to show the level of agreement between the target volume range and the delivered volume using the CPB and the NPB. Two-sided Student's t-tests were used to compare mean values, and chi-square tests were used to compare the proportions of the differences between the two groups. Two-sided p-values less than 0.05 were considered statistically significant.

### (3) Result

After the completion of testing procedures, each of the 730 values for TV and PIP that were produced by the CPB and the NPB were collected and analysed. Table 3 shows the mean delivered TVs and the accuracy of the delivery of the four target volume ranges. The proportion of accurate TV delivery for the 36-70 ml range was higher with the NPB than with the CPB (84.2% versus 45.9%; p<0.001). For the other target TV ranges, the proportion of accurate TV delivery by the NPB was higher than that obtained with the CPB (p<0.0001). The proportions of accurate TV for the NPB versus the CPB, respectively, were as follows: 93.2% versus 42.7% for 60ed with the CPB (p<0.00B and the NPB were collected and analysed or 144-210 ml, respectively

TABLE 3
Comparison of the accuracy of the delivery of the four target tidal volume (TV) ranges between the
conventional paediatric self-inflating bag (CPB) and the novel paediatric self-inflating bag (NPB).

| Body | Target TV range | Mean delivered TV (SD); ml | | No. of accurate delivering TV range (% of total 730 TV delivery) | | |
|---|---|---|---|---|---|---|
| weight | (6-7 ml/kg) | CPB | NPB | CPB | NPB | p-value |
| 6-10 kg | 36-70 ml | 63.8 (30.0) | 65.0 (12.3) | 335 (45.9) | 615 (84.2) | <0.001 |
| 10-15 kg | 60-105 ml | 95.4 (35.9) | 88.8 (13.6) | 312 (42.7) | 680 (93.2) | <0.001 |
| 15-24 kg | 90-168 ml | 123.7 (36.6) | 129.2 (19.5) | 513 (70.3) | 701 (96.0) | <0.001 |
| 24-30 kg | 144-210 ml | 167.3 (37.2) | 164.2 (23.0) | 457 (62.6) | 666 (91.2) | <0.001 |

SD: standard deviation

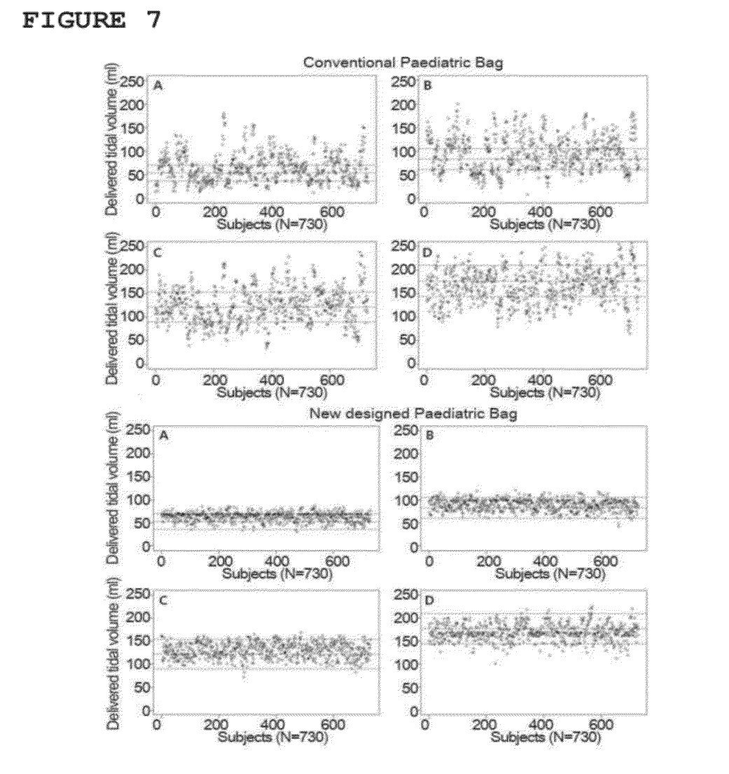

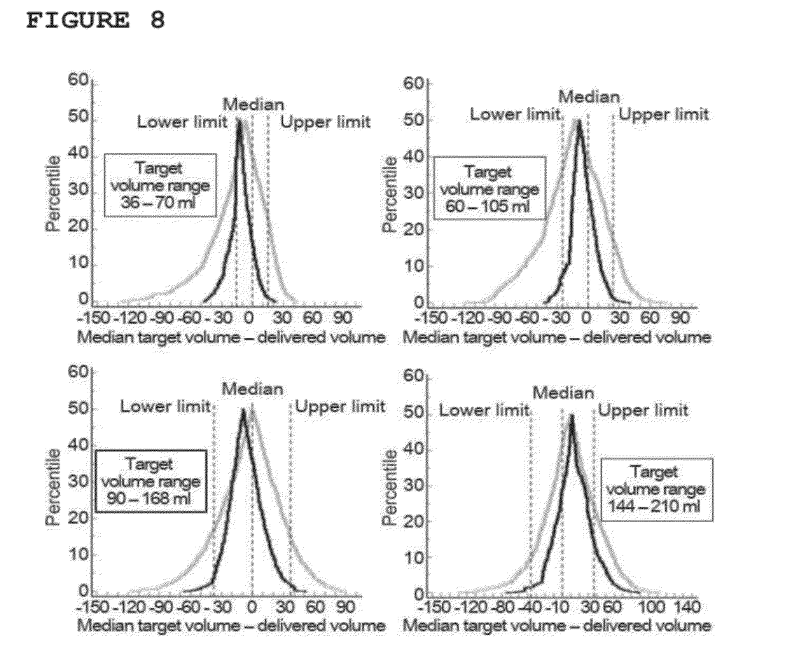

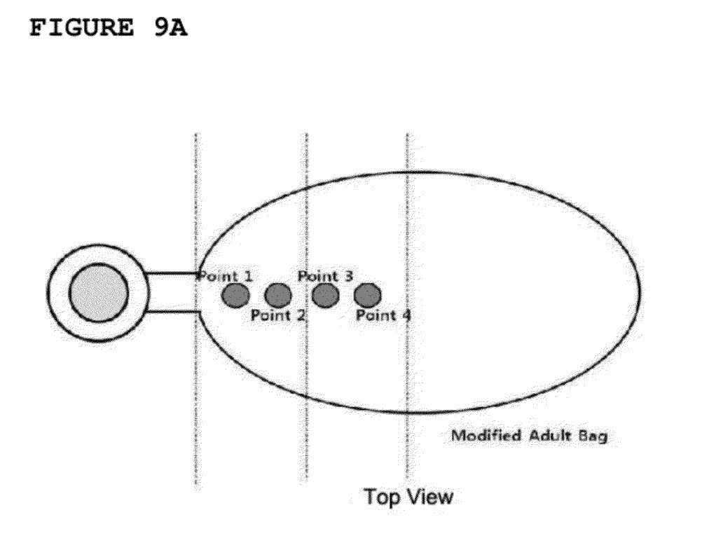

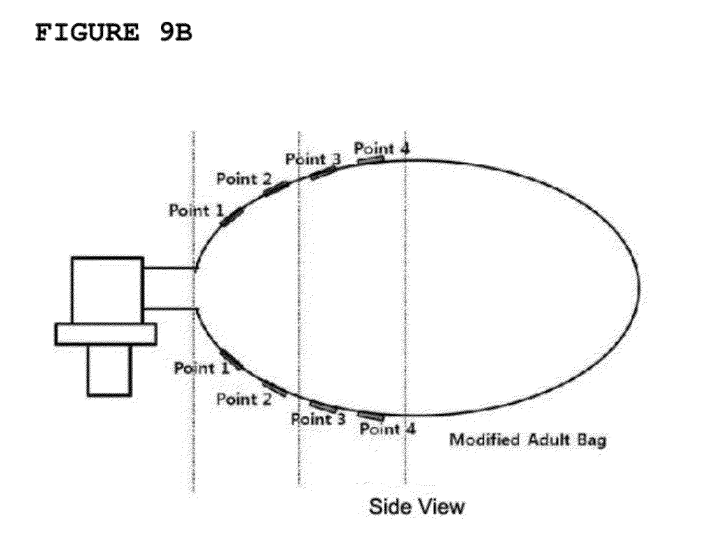

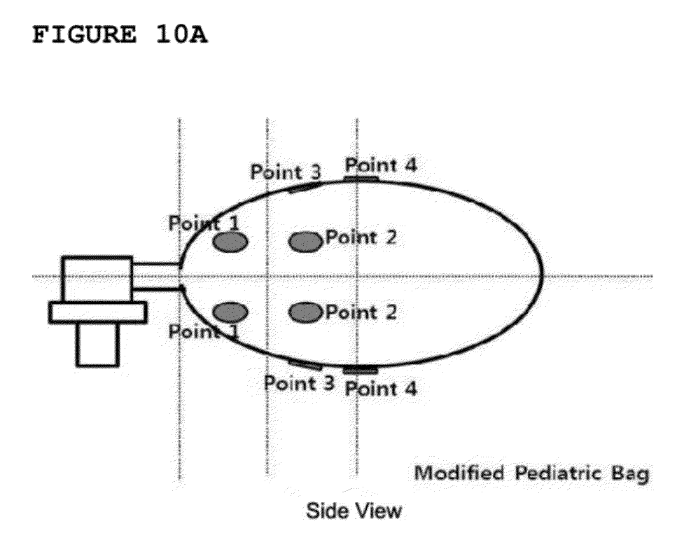

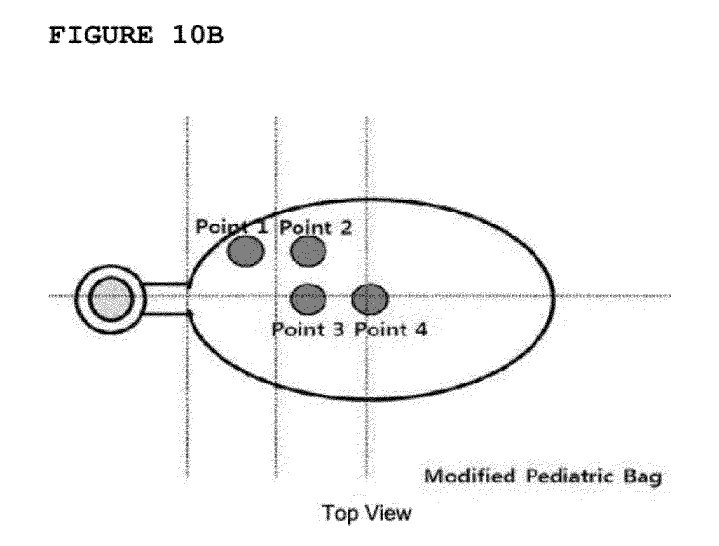

In comparison to the NPB, the CPB showed a more varied distribution of TVs in all four target TV ranges and a higher frequency of delivery of TVs that were outside of the target TV range (FIG. 7, FIG. 8). FIG. 8 shows the narrow-ranged distribution of the TVs delivered by the NPB compared to the CPB in all four target TV ranges. TVs delivered by the NPB tended to be at the higher end of the target TV ranges (from the median to the upper limit); this pattern is seen most clearly in the data for the 36-70 ml range. The one exception to this pattern was the data distribution for the NPB-administered TV for the 144-210 ml range. This range tended to include primarily administered TVs in the lower range (FIG. 8).

For the four target TV ranges, there were no significant (less than 0.5 cm Hg) differences in PIP between the CPB and NPB (Table 4).

TABLE 4
Comparison of the mean peak inspiratory pressure of the four target tidal
volume (TV) ranges between the conventional paediatric
self-inflating bag (CPB) and the novel paediatric self-inflating bag (NPB).

| | | Mean peak inspiratory | | |
|---|---|---|---|---|
| | Target TV range | pressure (SD); cmHg | | |
| Body weight | (6-7 ml/kg) | CPB | NPB | p-value |
| 6-10 kg | 36-70 ml | 3.74 (1.77) | 3.97 (0.86) | 0.002 |
| 10-15 kg | 60-105 ml | 5.49 (2.15) | 5.39 (1.03) | 0.244 |
| 15-24 kg | 90-168 ml | 7.21 (2.34) | 7.69 (1.39) | <0.001 |
| 24-30 kg | 144-210 ml | 9.77 (2.44) | 9.79 (1.73) | 0.803 |

SD: standard deviation

### (4) Conclusion

In conclusion, to supply the proper ventilation to small children of varying sizes in pediatric resuscitation, a narrowed and accurate range of TV delivery is required. The NPB will be a very useful device for delivering more accurate TVs for various target volume ranges in pediatric resuscitation and respiratory emergency situations.

1. A self-inflating bag for pediatric and adolescent resuscitation providing various different range of accurate tidal volume depending on different size or weight of patient by marking compression points on the self-inflating bag.

2. The self-inflating bag according to claim 1, wherein in the self-inflating adult bag, equally dividing the bag into five parts from the start point to mid-point of the bag and setting point 1, point 2, point 3 and point 4 in order except mid-point on the surface of the bag from a respiratory valve of the bag, thereby providing 100-200 ml tidal volume at point 1 near the valve, 200-300 ml tidal volume at point 2, 300-400 ml tidal volume at point 3, and 400-500 ml tidal volume at point 4.

3. The self-inflating bag according to claim 1, wherein in the self-inflating pediatric bag, equally dividing the bag into four parts from the start point to mid-point of the bag and setting point 1, point 2, point 3 and point 4 in order on the surface of the bag from a respiratory valve of the bag, thereby providing 36-70 ml tidal volume at point 1 near the valve, 60-105 ml tidal volume at point 2, 90-168 ml tidal volume at point 3, and 144-210 ml tidal volume at point 4.

4. The self-inflating bag according to claim 1, wherein the compression points are engraved in the surface of the bag or are embossed on the surface of the bag not to be slipped.

5. A method providing accurate tidal volume depending on different size or weight of pediatric and adolescent patients comprising:
preparing a self-inflating adult bag with specific compression marks on the surface of the bag and providing 100-200 ml tidal volume at point 1 near the valve, 200-300 ml tidal volume at point 2, 300-400 ml tidal volume at point 3, and 400-500 ml tidal volume at point 4, by equally dividing the bag into five parts from the start point to mid-point of the bag and setting point 1, point 2, point 3 and point 4 in order except mid-point on the surface of the bag from a respiratory valve of the bag in the self-inflating adult bag.

6. The method according to claim 5, wherein in the self-inflating pediatric bag, providing 36-70 ml tidal volume at point 1 near the valve, 60-105 ml tidal volume at point 2, 90-168 ml tidal volume at point 3, and 144-210 ml tidal volume at point 4 by equally dividing the bag into four parts from the start point to mid-point of the bag and setting point 1, point 2, point 3 and point 4 in order on the surface of the bag from a respiratory valve of the bag.

7. The method according to claim 5, wherein the compression points are engraved in the surface of the bag or embossed on the surface of the bag not to be slipped.

8. The method according to claim 5, wherein the method further comprising:
placing thumb at the upper points of the bag and index or middle finger at the lower points of the bag; and compressing the bag until the upper and lower points meet one another over 1 sec, to deliver preset target volumes.
